(19) United States
(12) Patent Application Publication
(10) Pub. No.: US 2011/0238432 A1
(43) Pub. Date: Sep. 29, 2011

(54) METHOD AND SYSTEM FOR OPTIMIZED DISTRIBUTION AND ADMINISTRATION OF VACCINATIONS

(75) Inventor: Casey DeLoach, Orlando, FL (US)

(73) Assignee: VAXCARE CORPORATION, Orlando, FL (US)

(21) Appl. No.: 12/732,067

(22) Filed: Mar. 25, 2010

(51) Int. Cl. G06Q 50/00; G06Q 10/00

(52) U.S. Cl. 705/2

(57) ABSTRACT

The invention is directed to a method and system of distributing vaccination carts comprising the steps of entering into an optimization module which includes entering the type of immunization desired to be distributed, identifying the goals of a vaccination program, and running an algorithm to determine the proper number of vaccination shots to provide the Provider; operating a service module to create a master schedule to allocate the distribution cart to one or more Providers; running an issue detection module to detect whether a networked computer determines if there are any unacceptable sensor readings; and determining whether a sensor detects an issue and, if so, returning to the optimization module.

### BACKGROUND OF THE INVENTION

1. Field of the Invention

The present invention relates to a computer-implemented method and computerized system for ordering, allocating, distributing, administrating, and processing vaccinations (as well as related information, insurance reimbursement forms, etc). In addition, the invention relates to a computer-implemented method for optimizing distribution and administration of vaccinations reducing the overall costs, increasing efficiency, and ensuring the integrity, effectiveness and viability of vaccinations.

2. Description of the Related Art

Over the past century, advances in medicine have given rise to numerous vaccinations to prevent diseases. For example, vaccinations for once life threatening diseases such as small pox, polio, tuberculoses and typhoid have been developed. These advances in medicine have not only improved the quality of life, but also greatly increased the average life expectancy.

Modern pharmaceutical companies (hereinafter “Pharmaceuticals”) have also made great strides in their ability to create vaccinations for influenza, commonly referred to as “the flu.” This has included the rapid isolation and creation of vaccines for newly discovered strains of the flu. Once these new strains are recognized, Pharmaceuticals can now effectively create large quantities of vaccinations each year for these new strains of the flu.

While pharmaceuticals can now efficiently create vaccinations for a new strain of the flu, very little has been done to create effective channels and techniques for distributing these important inoculants. Traditionally, flu vaccinations were distributed directly to facilities such as doctor's offices, medical clinics and hospitals, for administration of a range of vaccines, including flu shots. However, these facilities had difficultly accurately forecasting the precise quantity of vaccine doses to order and delivering them to patients in a cost effective and efficient manner.

The difficultly in forecasting the precise quantity of vaccines to order resulted in unused quantities of vaccinations and spoilage of these often scarce medicines. Over time, the inability to forecast vaccination needs resulted in a chain reaction of events that lead to higher costs and lower revenues for vaccine providers and lower utilization rates for patients. The pattern started when vaccine providers would order conservative quantities of vaccine in an effort to reduce spoilage and waste. However, the conservative ordering also increased the risk that a patient would visit their provider, but be unable to receive a vaccine. Over time, patients became apathetic about visiting a provider to receive a vaccine for flu (or other preventable disease). Today's vaccine market is marked by both low-profit margins for providers and low utilization rates for patients.

Because of this inability to forecast the correct amount of vaccine doses and patient apathy, many doctors have opted not to offer administration of flu shots. In short, it was no longer financially viable for traditional medical clinics and family doctors to offer these important medicines.

Within the last decade, alternative facilities have begun to offer centralized administration of flu shots and other vaccines. These alternatives include public health administrations (PHAs) of municipal governments—which have set up flu shot booths at fire stations and other city facilities, such as city hall, schools, recreation centers, and the like. In addition, large pharmacies and retailers, such as CVS and Walgreens, now offer flu shots.

While vaccination offerings by PHAs and retailers have addressed some of the cost and spoilage issues raised by the prior operating model, these alternative facilities aggravate some of the Provider service issues that reduce a patient's willingness to receive a vaccine. First and foremost, the use of alternative facilities for administration of vaccines has reduced the ability for patients to receive ancillary and/or comprehensive treatment (such as yearly medical check-ups, blood work, etc.) with their regular family doctors. In addition, the limited number of alternative facilities has caused long wait times to receive the vaccine shots, which reduced patient incentives for obtaining yearly treatments.

The alternative facilities also create a new complication in that the alternative vaccine providers often do not have formal medical training or provide medical services on a regular basis. Because of this, there is an increased risk that the vaccine will not be properly maintained and preserved in a refrigerated condition—and may not be correctly administered.

Finally, these alternative facilities are not equipped to provide accurate processing of insurance claims regarding vaccine administration. This results in patients either not receiving reimbursement for vaccinations, or having to process the potentially complicated forms themselves. This creates yet another potential obstacle to increase patient demand.

Accordingly, there is a need in the art of vaccine distribution for an intermediary between manufacture of flu shots (and other vaccines) and their administration. This new component should act as a “facilitator” that ensures proper allocation and viability of vaccines, giving providers a turnkey solution to their need for flu shots without risk of over or under ordering. Moreover, such facilitator should afford the ability for improved vaccine and patient information quality control, improved patient awareness and education, shorter wait times and lower pricing for vaccinations.

### SUMMARY OF THE INVENTION

In view of the foregoing background, the present invention solves the limitations found in current distribution and administration systems for vaccinations. Moreover, this invention creates a “facilitator” that bridges the manufacture of vaccinations with administration by medical clinics, doctors, retailers and PHAs (collectively, “Providers”). This turn-key solution affords enhanced quality control, lower costs, shorter wait times for vaccine administration, and higher vaccination rates. Moreover, it creates a trusted source for vaccine administration by creating more consist reliable vaccination shot delivery through ensuring the integrity and viability of each vaccination.

According to an embodiment of the present invention, a computer-implemented method of optimizing, distributing and allocating vaccination shots includes: receiving Provider information for a Provider; receiving the type of vaccine to be administered; reviewing a Provider profile and settings for the Provider; executing an algorithm to approximate the number of vaccination shots to deliver to the Provider based on the type of vaccine to be administered and the Provider profile and settings for the Provider; and creating a schedule to deliver the vaccination shots to the Provider. Creating a schedule can include: identifying the number of Provider facilities to distribute vaccination shots to; determining the delivery schedules for each identified Provider facility; and specifying that a vaccination cart is to be provided.

According to an embodiment of the present invention, a Provider user account is created that includes the Provider information, wherein the Provider information includes a unique user id and password, a proper Provider level, and profile settings.

According to an embodiment of the present invention, notification that a Provider terminal has determined that there is an unacceptable reading for a sensor on the vaccination cart is received.

According to an embodiment of the present invention, information regarding the number of remaining vaccination shots left in the refrigeration unit is received.

According to an embodiment of the present invention, information regarding any viability issues which may affect the integrity of the vaccination shots is received.

According to an embodiment of the invention, a server for optimizing, distributing and allocating vaccination shot includes a processor operable to execute computer program instructions, a memory operable to store computer program instructions executable by the processor, and computer program instructions stored in the memory and executable to perform the above-described method.

### BRIEF DESCRIPTION OF THE DRAWINGS

The details of the present invention, both as to its structure and operation, can best be understood by referring to the accompanying drawings, in which like reference numbers and designations refer to like elements.

FIG. 1A is an exemplary block diagram of a network system in which the present invention may be implemented according to an embodiment of the invention.

FIG. 1B is an exemplary block diagram of a computer system, such as a server, according to an embodiment of the invention.

FIG. 2 is an exemplary flow-chart of a process for ordering, allocating and distributing the proper amount of vaccination according to an embodiment of the invention.

FIG. 3 is an exemplary flow-chart of a process for creating user accounts according to an embodiment of the present invention.

FIG. 4 is an exemplary flow-chart of a process for optimizing distribution of vaccines according to an embodiment of a present invention.

FIG. 5 is an exemplary flow-chart of a process that identifies the preferred steps for the service and diagnostic module.

FIG. 6 is a flow-chart that identifies the preferred steps for the issue detection module.

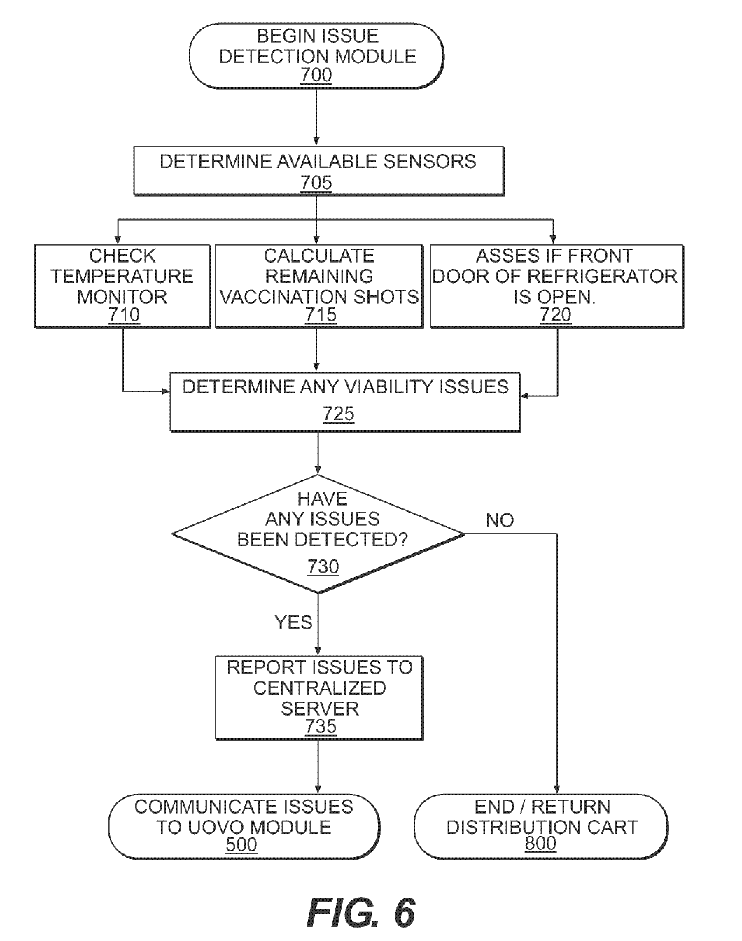

FIG. 7 is a front view of an exemplary vaccination cart according to an embodiment of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

A computer-implemented method and system for optimizing distribution and administration of vaccinations to reduce the overall costs and ensure the integrity, effectiveness and viability of the vaccine is provided. A “facilitator” is created that bridges the manufacture of vaccinations with administration by medical clinics, doctors, retailers and PHAs. This turn-key solution affords enhanced quality control, shorter wait times for vaccine administration, and lower costs and higher vaccination rates. Moreover, it creates a trusted source for vaccine administration by creating more consist reliable vaccination shot delivery through ensuring the integrity and viability of each vaccination.

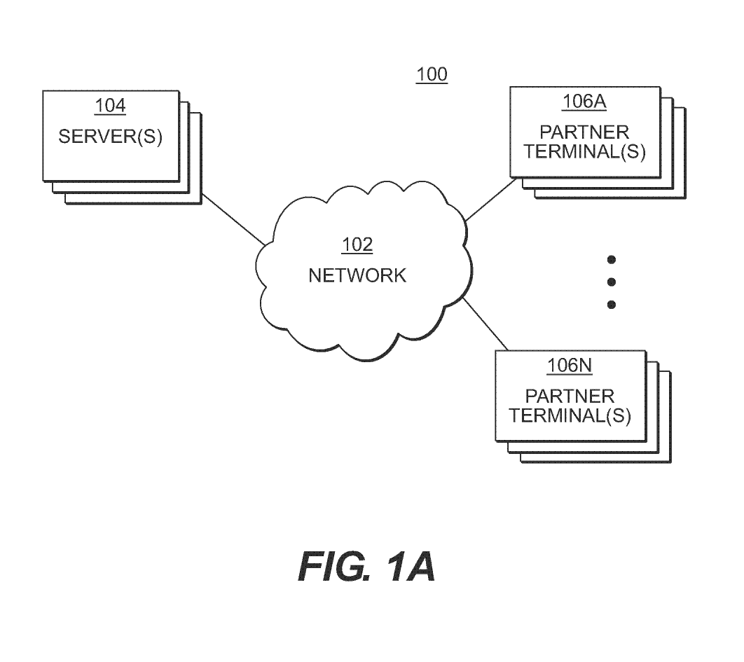

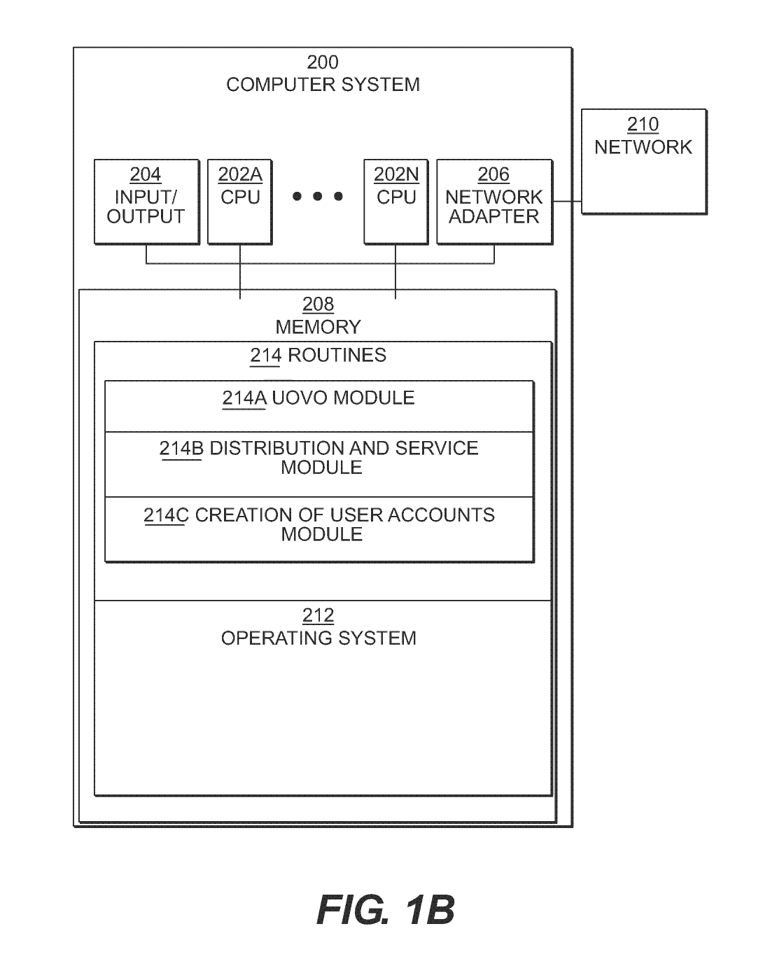

As an example, such a turn-key solution may be provided using a network system 100 such as that shown in the FIG. 1A embodiment of the present invention. FIG. 1A shows a network 102, one or more Servers 104, and a plurality of Provider Terminals 106A-N. Network 102 includes any communications network that is now in service or which may be developed in the future. Network 102 includes, but is not limited to, one or more public or private communications networks, such as the Internet, wired or wireless telephone networks, wired or wireless data networks, local area networks, etc. Servers 104 are provided by a “facilitator,” which is an entity in the business of bridging the manufacture of vaccinations with administration by medical clinics, doctors, retailers and PHAs. Server 104 performs the functions of the present invention which optimize distribution and administration of vaccinations as describe more fully herein below. Provider Terminals 106A-N are any type of device that requests the services provided by servers 104. Such devices may include personal computers, Web-enabled televisions, mobile telephones and PDAs, and the like. In an embodiment of the present invention, a Provider terminal is operable to implement an issue detection module as described more fully herein below.

An exemplary block diagram of a computer system 200, such as Servers 104, is shown in FIG. 1B. System 200 is typically a programmed general-purpose computer system, such as a personal computer, workstation, server system, minicomputer, or mainframe computer. System 200 includes one or more processors (CPUs) 202A-202N, input/output circuitry 204, network adapter 206, and memory 208. CPUs 202A-202N execute program instructions in order to carry out the functions of routines 214 for creating user accounts, ordering, allocating and distributing the proper amount of vaccination, optimizing distribution (i.e., scheduling the distribution) of vaccines, and issue detection according to an embodiment of the present invention as described more fully herein below. Typically, CPUs 202A-202N are one or more microprocessors, such as an INTEL PENTIUM® processor. FIG. 1B illustrates an embodiment in which System 200 is implemented as a single multi-processor computer system, in which multiple processors 202A-202N share system resources, such as memory 208, input/output circuitry 204, and network adapter 206. However, the present invention also contemplates embodiments in which system 200 is implemented as a plurality of networked computer systems, which may be single-processor computer systems, multi-processor computer systems, or a mix thereof.

Input/output circuitry 204 provides the capability to input data to, or output data from, database/system 200. For example, input/output circuitry may include input devices, such as keyboards, mice, touchpads, trackballs, scanners, etc., output devices, such as video adapters, monitors, printers, etc., and input/output devices, such as, modems, etc. Network adapter 206 interfaces device 200 with network 210. Network 210 includes any communications network that is now in service or which may be developed in the future. Such a network may include one or more public or private communications networks, such as the Internet, wired or wireless telephone networks, wired or wireless data networks, local area networks, etc.

Memory 208 stores program instructions that are executed by, and data that are used and processed by, CPU 202 to perform the functions of system 200. Memory 208 may include electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra direct memory access (UDMA), or a small computer system interface (SCSI) based interface, or a variation or enhancement thereof, such as fast-SCSI, wide-SCSI, fast and wide-SCSI, etc, or a fiber channel-arbitrated loop (FC-AL) interface, or Serial AT Attachment (SATA), or a variation or enhancement thereof.

The content of memory 208 varies depending upon the function that system 200 is programmed to perform. Operating system 212 provides overall system functionality. In the FIG. 1B embodiment of the present invention, Routines 214 includes UOVO module 214A, distribution and service module 214B, and creation of user accounts module 214C.

As shown in FIG. 1B, the present invention contemplates implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

It is important to note that while the present invention has been described in the context of a fully functioning data processing system, those of ordinary skill in the art will appreciate that the processes of the present invention are capable of being distributed in the form of a computer readable medium of instructions and a variety of forms and that the present invention applies equally regardless of the particular type of signal bearing media actually used to carry out the distribution. Examples of computer readable storage media include, floppy disks, hard disk drives, CD-ROMs, DVDROMs, RAM, flash memory, etc.

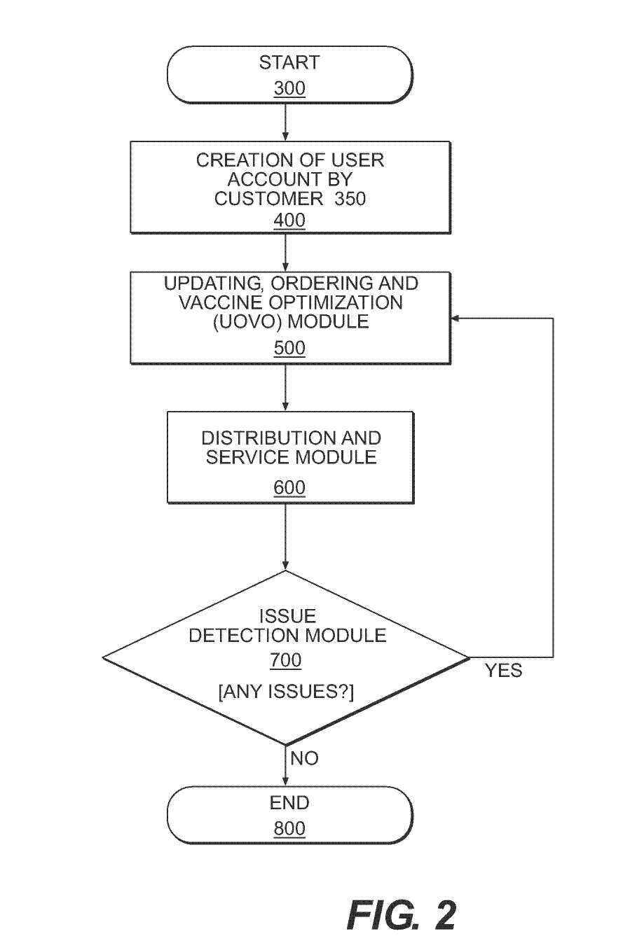

An exemplary flow-chart of a process for ordering, allocating and distributing the proper amount of vaccination according to an embodiment of the present invention is shown in FIG. 2. In this embodiment, the method starts (at step 300) when a Provider 350 logs onto a server, such as server 104 over a computer network, such as the internet via a Provider terminal, such as Provider terminal 106, having a web based interface (at step 400). The act of the Provider 350 logging into the network can either be to establish a Provider profile or to simply access the Provider's account. Other access systems can allow Provider access through an iPhone, Blackberry or other type of cellular telephone, PDA, other similar handheld device. The Provider 350 can also access account information through calling a dedicated phone number.

Upon creation of (or logging into an existing) user account using account creation module 214C, the Provider 350 is then prompted to enter (or update) information in a variety of fields located in an updating, ordering and vaccine optimization (“UOVO”) module 214A (at step 500). In the FIG. 2 embodiment of the present invention, the UOVO module 214A includes an algorithm that gauges the proper amount of vaccination shots to distribute to a Provider 350. In addition, the UOVO module 214A can, if requested, analyze other Provider 350 vaccination needs and orders to determine a proper schedule to distribute the vaccination shots. In an embodiment of the present invention, the vaccination shots are provided to a Provider along with a vaccination cart 900 to facilitate the proper maintenance and presentation of the vaccination shots. In an embodiment of the present invention, the vaccination cart 900 may include a Provider terminal operable to access the server implementing the functions of the present invention, or such access may be provided via a standalone terminal otherwise available to the Provider.

After the UOVO module 214A analyzes the proper amount of vaccination shots to distribute to the Provider accessing the server, the method next includes a distribution and service module 214B (at step 600). This service module 214B sets up a schedule when to distribute the vaccination shots. In the FIG. 2 embodiment of the present invention, the service module 214B can set up a master schedule where a vaccination cart 900 provided with vaccination shots is delivered to one facility or Provider 350 and then later moved to a second facility or Provider 350 to maximize vaccine distribution. In an embodiment of the present invention, a vaccination cart 900 placed at a Provider 350 facility includes an issue detection module stored within the Provider terminal 106 that monitors a variety of sensors provided on vaccination cart 900 to determine if there is any risk to the viability and integrity of the vaccination shots. Should any issue be detected, a communication is sent wirelessly from the computer 106 to sever 104 over network 102.

Any detected issue is sent to the UOVO module (at step 500) to be interpreted by a detection module on a Provider terminal. The UOVO module can order a secondary shipment of vaccination shots to be sent out to the Provider 350 location. Alternatively, the UOVO module can direct an additional distribution cart 900 to be sent to the Provider 350.

As is further shown in FIG. 2, if there is no irregularity found by the issue detection module the vaccination cart 900 remains in continuous use until (a) all of the vaccination shots are spent, (b) the vaccination cart 900 is replenished with additional vaccination shots, or (c) the vaccination cart 900 is retrieved from the Provider 300 (at step 800). More specifically, the vaccination cart 900 can either be returned to re-processed for a subsequent distribution or alternatively can be directly relocated to another Providers 350 if found to contain a sufficient batch of vaccination shots. Although the invention certainly contemplates the ability to deliver the vaccination cart 900 along multiple facilities and Providers, it is preferable that each Provider 350 be allocated its own dedicated vaccination cart 900.

The process illustrated in FIG. 2 creates a “facilitator” of vaccine—acting as an effective distribution channel between the vaccine manufacture and the end administrator (i.e., the Provider 350). Moreover, this facilitator service is at no cost to the Provider 350—as the facilitator is directly paid by the patient 200. Instead, the Provider 350 is compensated by the facilitator (for example, based upon a portion of the payment by the patients 200). This not only creates a no-risk solution for these various medical providers, but motivates them to again offer vaccination services at their respective medical facilities.

Creation of a Provider User Account

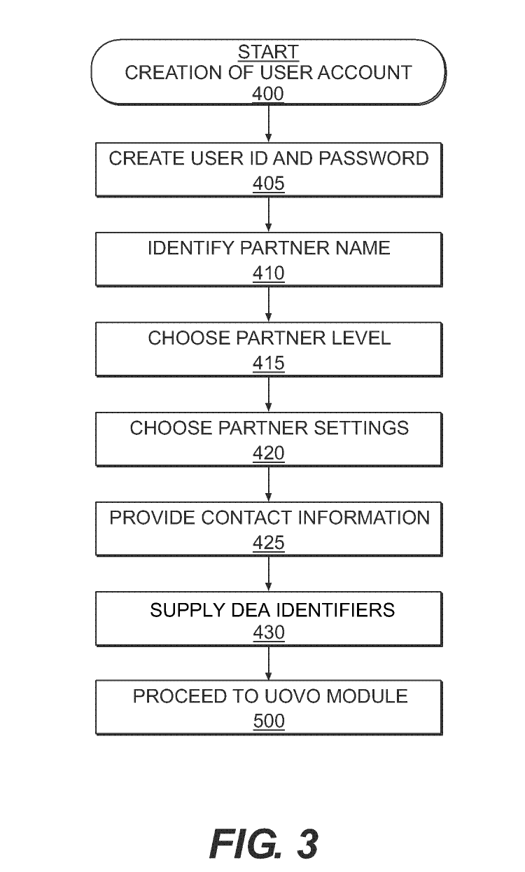

A flow-chart of a process for creating a Provider user account on a graphical user interface (GUI) accessible through a dedicated secure server according to an embodiment of the present invention is shown in FIG. 3. This account creation module 400 begins with creation of a unique user id 401 and a confidential password 402 (a step 405) by the Provider 350. These log-ins help identify the Provider 350 each time they log into the system to either order an initial supply of vaccination shots, order a vaccination cart, request that another (additional or replacement) vaccination cart be positioned at a facility, order additional vaccination shots, to determine the amount of vaccination shots that have been used, ordered or available to that Provider 350 or a combination thereof. In addition, the Provider 350 should enter a Provider name (at set 410) including proper contact information. This is because one Provider 350 may have several facilities in which to distribute vaccinations.

Next, the Provider 350 must select (at step 415) a Provider level. This Provider level corresponds to the type of entity of the Provider 350 including, but not limited to, a PHA, a municipality, a school, a retailer, a business, and the like, as well as the overall size and number of locations (facilities, branches, schools, etc.) for that Provider 350. Next, the Provider 350 should choose Provider/profile settings (at 420) that correspond to the underlying demographics of the likely patient pool (pregnant women, infants, children, the elderly, etc.), vaccination priorities, goals, strategies, and other related reasons for providing inoculation services. These Provider settings serve a very important role in allocating and servicing a Provider 350. For example, the demographics provided in a setting may determine the dose of the vaccination (one may differ for children compared to adults) or the priority to distribute a particular vaccination (for example, the H1N1 vaccine should first be given to children and military personnel not the elderly). Accordingly, Provider settings for a PHA providing flu shots may be very different than a private nursing home providing other vaccinations for elderly patients.

As further shown in the FIG. 3 embodiment of the present invention, the Provider 350 should also provide detailed contact information (at 425) that includes a key emergency contact person, email and SMS texting information—sufficient to provide an alert should any emergency occur. This key emergency contact should be for the Provider 350—not just an individual branch or location for that Provider 350. In addition to contact information, any applicable Drug Enforcement Administration (DEA) identifiers should be provided (at step 430) if applicable. Once this information is provided, the Provider 350 is able to access, re-access, log-in, update and use the UOVO module (at step 500).

Optimized Distribution of Vaccine

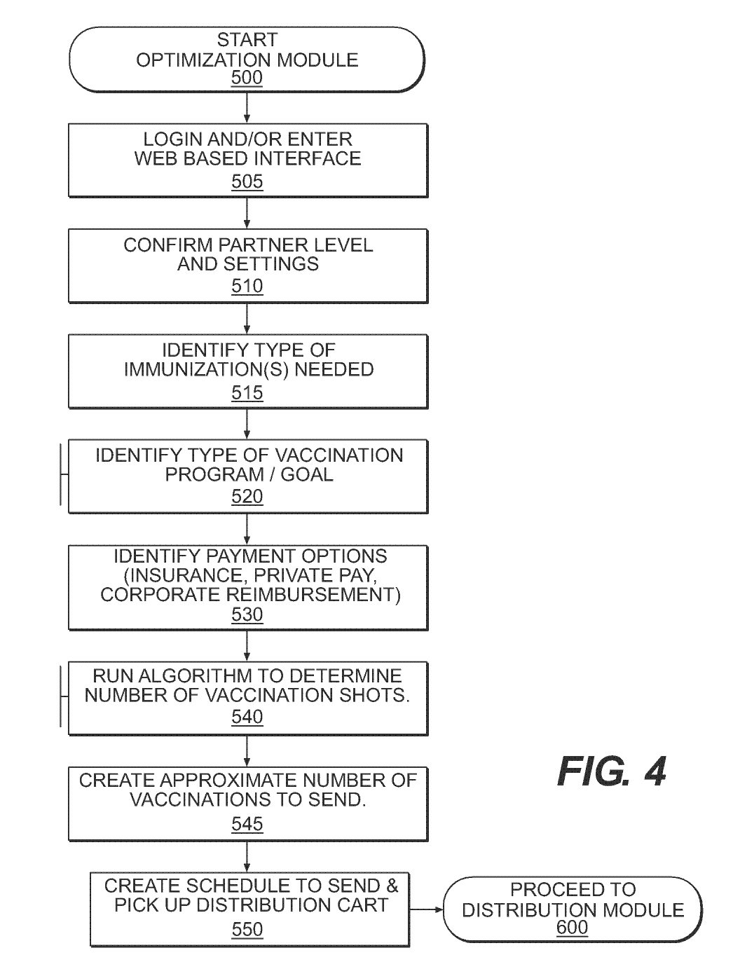

A flow-chart of the process performed by the UOVO module 500 for optimizing distribution and allocation of vaccination shots according to an embodiment of the present invention is shown in FIG. 4. It is important to note the UOVO module 500 operates to ensure efficient distribution of a variety of vaccination shots, which includes, but is in no way limited to flu shots.

As shown in the FIG. 4 embodiment of the present invention, the optimization module 500 begins by requiring the Provider 350 to login and enter the web-based portal (at step 505) provided by server. This web-based portal is available through a graphical user interface (GUI) accessible through the Internet. Once the Provider enters their user ID and password, they are asked to confirm (at step 510) their Provider level (i.e., they are a municipality, etc.) as well as profile settings.

As further shown in the FIG. 4 embodiment of the present invention, the Provider 350 is prompted to identify the type of immunization needed (at step 515). This can include identification of the need to provide flu shots, as well as other immunizations including, but not limited to, pneumonia, tetanus, hepatitis A, or a combination of such vaccines. After receiving the type of vaccine to be administered, the Provider 350 is prompted (at step 520) to enter a variety of goal specific information regarding the Provider's necessity and basis for seeking quantities of vaccination shots.

Information used to identify these goals includes, but is not limited to, listing the number of facilities that will administer vaccination shots, identifying acceptable payment options including, but not limited to, insurance, private pay, corporate reimbursement, and providing when each facility is available to administer vaccinations shots. Demographics cross referenced through review of the profile settings for average age, sex, income level, and the like can also be reviewed. Based upon the aggregate of this information, an algorithm is run (at step 540) to approximate the correct number of initial and future vaccination shots needed by the Provider 350 for each facility. Factors that are considered by the algorithm include the Provider's specific vaccination administration history including, but not limited to, the number and type of vaccination shots administered by the Provider over different time periods, the number of patients the Provider services, the total number of visits per patient, the demographics of the patients, the number of doctors within a specific geographic region, national shot projections, and southern hemisphere flu activity. These factors are also considered when determining a Provider's future delivery of vaccination shots.

Based upon running the algorithm shown in FIG. 4, a detailed report that includes an approximation (at step 545) of the number of vaccination shots to send the Provider 350 is generated. A proposed schedule for the delivery of the vaccination shots to the Provider 350 is then created (at step 550). In an embodiment of the present invention, the vaccination shots are delivered along with a vaccination cart to facilitate the maintenance and presentation of the vaccination shots. The schedule can be for distribution of vaccination shots to a single Provider 350, or a master schedule to send vaccination shots to a plurality of different Providers 350 to maximize use and access to a batch of vaccines. The protocol is then directed to the distribution and service module 600.

Distribution and Service Module

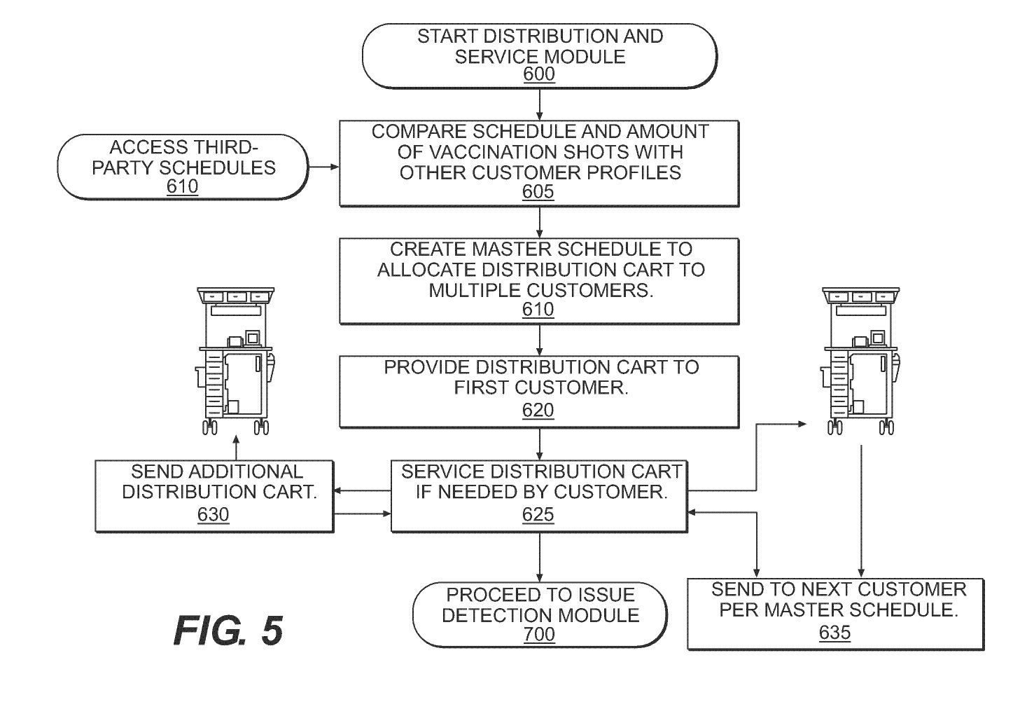

A flow-chart of the process performed by the distribution and service module 600 according to an embodiment of the present invention is shown in FIG. 5. The module begins by comparing (at step 605) the schedule created in the OUVO module (at step 550) with other recent Provider profiles and orders. This includes accessing (at step 610) the files of third-party Providers 350 and reviewing their schedules and previous orders (and underlying quantities) of vaccinations. Such access is required to determine, among other things, whether there are sufficient quantities of vaccination shots available for a particular vaccine, whether there is a vaccination cart available for distribution to a Provider 350 and if there are any vaccination carts 100 being returned in the near future. Based upon this comparison, a master schedule is created to allocate vaccination shots amongst one or more Providers. In an embodiment of the present invention, vaccination shots are delivered to Providers without a vaccination cart when the Providers have the requisite laboratory equipment to maintain and present the vaccination shots. In an embodiment of the present invention, the vaccination shots are distributed along with a vaccination cart dedicated to the respective Provider. In the embodiment of the present invention shown in FIG. 5, the master schedule is created such that a vaccination cart is delivered at specific times to multiple Providers 350.

Based upon the schedule, a vaccination cart is provided (at step 620) to an initial Provider 350. Vaccination shots that are maintained and presented using the vaccination cart can be recorded and identified on the server 104 such that there is end-to-end tracking of the vaccine batches from shipment to the facilitator, to distribution to the Provider 350 to administration to the patient. This ensures quality control as well as the ability to alert patients as well as Providers 350 should a quality control issue later become apparent.

If a Provider 350 requires service, they can place an alert via a Provider terminal 106 for a service technician to service the Provider. Such service (at step 625) can include, but is not limited to, delivering additional vaccination shots, replacing a broken vaccination cart 900, providing additional reimbursement forms, or providing medical equipment such as gauze, adhesive bandages, etc. Service can also include sending (at step 630) an additional distribution cart if the demand for vaccination is greater than previously expected.

Furthermore, based upon the master schedule, the distribution cart 900 can later be provided (at step 635) to a second Provider 350 for further distribution of vaccination shots. As with the initial Provider, the second Provider 350 will be provided the same types of services provided at step 625.

Issue Detection Module

An exemplary flow-chart of the process performed by issue detection module 700 according to an embodiment of the present invention is shown in Figure. Issue detection is provided in an embodiment of the invention where a vaccination cart 900 and Provider terminal 106 is provided to the Provider. The issue detection module begins by determining (at step 705) the total available sensors 126 connected to the Provider terminal 106 to provide self-diagnostics. As previously discussed, these sensors can provide a variety of diagnostic tests to ensure viability of the vaccination shots. These can include checking a temperature monitor (at step 710) to ensure the temperature has not risen above a pre-specific level which risks degradation of the vaccine, calculating (at step 715) the estimated remaining vaccination shots available (through counting the number of times the front door 161 has open and closed or alternatively by detecting the removal of vaccination shot from a vaccination tray hold the vaccination shots, as well as determining if the front door 161 has been opened an unreasonable amount of time (at step 720). Based upon measuring these various sensors, the Provider terminal 106 determines (at step 725) the viability of the stored vaccinations.

If any issues are detected (at 730) through the Provider terminal 106 interpreting data offered by the sensors, then a report is issued (at step 735) to the server 104. More specifically, these issues are communicated to the UOVO module 500. If there are no issues, then vaccination shots are simply distributed until the vaccination cart 900 is emptied or returned (at step 800) after the master schedule is complete.

The Vaccination Cart

In order to efficiently and effectively allocate the proper quantities of vaccine to a specific facility (such as a medical clinic, doctor's office, school, fire department, or other PHA facility), the use of an automated and networked vaccination cart 900 is contemplated in an embodiment of the present invention. This vaccination cart 900 should offer a “turn-key” solution to provide an all-in-one solution to distribute, store and administer vaccine (as well as process insurance reimbursement). Such a vaccination cart 900 can be used for any type of inoculation, including but not limited to flu shots.

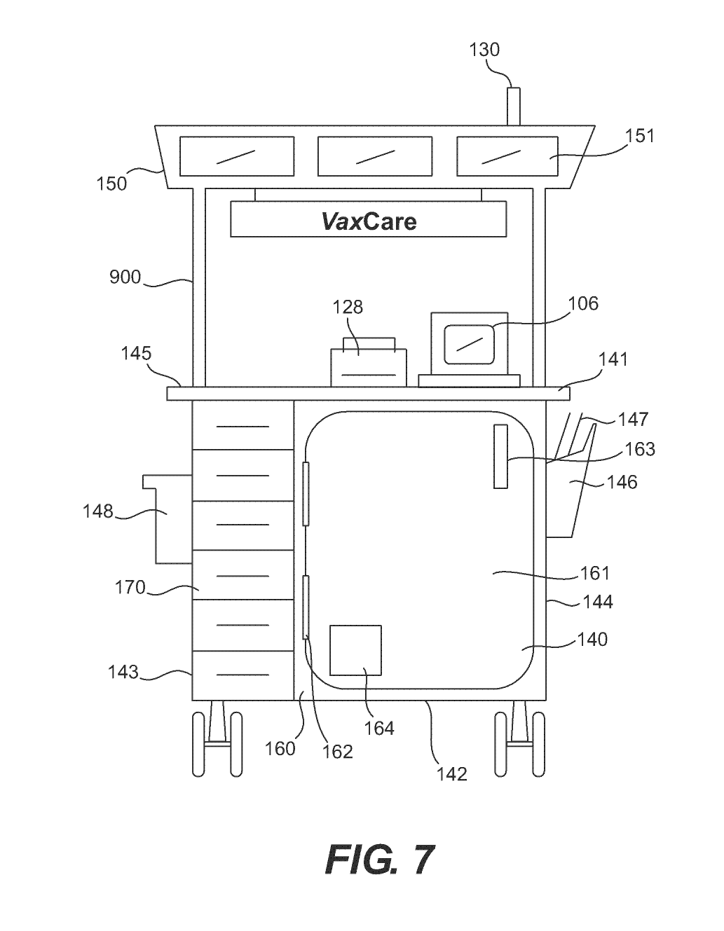

A vaccination cart 900 according to an embodiment of the present is shown in FIG. 7. In the FIG. 7 embodiment of the present invention, vaccination cart 900 is capable of storing and maintaining the one or more types of vaccination shots in a sterile, hygienic and refrigerated condition prior to administration to patients. In addition, the vaccination cart 900 performs self-diagnostics which ensure viability of the vaccination shots such as ensuring they have not fallen below or risen above a pre-specified temperature range. This self-diagnostic should include some form of a wirelessly networked Provider terminal 106, which can communicate with a server 104 (located at a remote location).

Although the vaccination cart 900 can take many a form, FIG. 1 offers one preferred embodiment of a robust all-in-one version. As illustrated in FIG. 1, the vaccination cart 900 includes a bottom portion 140 and a corresponding top portion 150. The bottom portion 140 includes various components to not only store vaccination shots (not shown), but to also maintain medical equipment for vaccine administration, as well as storage of pamphlets and paperwork regarding vaccination.

FIG. 1 further illustrates how the bottom portion 140 is essentially square or rectangular in shape and includes a top side 141, a corresponding bottom side 142, a left side 143 and a corresponding right side 144. On the top side 141 of the bottom portion 140 is a flat working table surface 145. This table surface 145 allows sufficient surface area to place various medical equipment (swabs, disinfectant, and adhesive bandages) prior to administration of a vaccination shot.

On the right side 144 (or alternatively the left side 143) of the bottom portion 140 is a vertical file sleeve 146. The file sleeve 146 is capable of storing various forms 147, including, but not limited to, as vaccine information sheets, custom consent forms, patient receipts, information regarding post-treatment after vaccination, health tips and insurance reimbursement forms.

The custom consent form 147 can be generated on site through use of the networked Provider terminal 106 and connected printer 128 in communication with the server 104 (located at a separate central location). The inquiries (i.e., spaces) provided in the custom consent form 147 can be populated and created by information stored on the server 104 through a Provider's 350 Provider level 415 and Provider settings 420. For example, the networked computer 106 can create one consent form for flu shots at a school, while create a separate and distinct form when giving another type of vaccination at a nursing home—all based upon the demographics inherent in the Provider settings 420.

In addition, through access with the Provider level 415 and other Provider 350 information stored on the central server 130, the custom consent form 147 can include a bar code or other alpha-numeric identifier that can help track vaccine administration (including but not limited to which patent was given a particular batch of vaccination shots). Such tracking capabilities can also assist in vaccine administration, pick-up and later insurance claims processing. The left side 143 (or alternatively the right side 144) of the bottom portion 140 includes a medical waste container 148 sufficient to discard used vaccination shots.

As further illustrated in FIG. 1, the bottom portion 140 includes a refrigeration unit 160 and a series of drawers 170. The refrigeration unit 160 can include an electric powered condenser sufficient to maintain a steady temperature for storage of vaccination shots in accordance with manufacturer specifications. In the alternative, the refrigeration unit 160 can simply be an insulated container that includes a refrigeration packet such as ice or dry ice. Regardless, the refrigeration unit should include a front door 161 sufficient to open and retrieve vaccination shots. This front door 161 can be opened through a series of rotatable hinges 162. In addition, the front door 161 can include a front handle 163.

Located on the front door 161 is a temperature meter 164. This temperature meter 164 can be as simple as a solution of glycol, a digital thermometer, or a standard thermometer. It is preferable, but not necessary, for the temperature meter 164 to provide a warning to indicate whether the inside temperature of the refrigerator 160 has fallen below a pre-determined level—indicating a risk as to the integrity and viability of the stored vaccination shots.

Affixed on top of the table surface 145 is the computer 106. The computer 106 can be connected to various sensors (not shown) located on the vaccination cart 900. These sensors can include the temperature meter 164, as well as other devices capable of measuring when the various drawers are opened and closed and/or the number of vaccination shots removed from the refrigerator unit 160. This information can then be interpreted by the networked computer 106 and potentially relayed to the centralized server 130 via a wireless antenna 127.

A printer 128 can be attached to the networked computer 125. The printer 128 can be used to print out paperwork for patients 200 such as the custom consent form 147. In addition, the printer 128 can be used to create a record of the vaccinations stored within the vaccination cart 900 for later processing. In addition, a self-contained power source (not shown) can be located within the bottom portion 140 capable of powering the refrigeration unit 160, the computer 106, the printer 128 and various sensors.

In addition to the lower portion 140, FIG. 1 illustrates the upper portion 150 of the vaccination cart 900. As shown, the upper portion 150 includes a plurality of clear bays 151 which can store a variety of equipment and supplies. The upper portion 150 can further include a hand sanitizer dispenser 152. In addition, the upper portion 150 can include a sign 152 sufficient to alert patients of the vaccination services being offered.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

I claim:

1. A computer-implemented method of optimizing, distributing and allocating vaccination shots, comprising the steps of:
(a) receiving Provider information for a Provider;
(b) receiving a type of vaccine to be administered;
(c) reviewing a Provider profile or settings for the Provider;
(d) executing an algorithm to approximate the number of vaccination shots to deliver to the Provider based on the type of vaccine to be administered and the Provider profile or settings for the Provider; and
(e) creating a schedule to deliver the vaccination shots to the Provider;

2. The method of claim 1, further comprising the step of:
creating a Provider user account including the Provider information, wherein the Provider information includes a unique user id and password, a proper Provider level, and choosing profile settings.

3. The method of claim 2, further comprising the step of supplying the proper DEA identifiers.

4. The method of claim 1, wherein the step of creating a schedule further includes the step of:
identifying the number of Provider facilities to distribute vaccination shots to.

5. The method of claim 4, wherein the step of creating a schedule further includes the step of:
determining the schedules for each identified Provider facility.

6. The method of claim 5, wherein the step of creating a schedule further includes the step of:
specifying that a vaccination cart is to be provided along with the vaccination shots.

7. The method of claim 6, wherein the step of creating a schedule further includes the step of:

8. The method of claim 6, further comprising receiving notification that a Provider terminal has determined that there is an unacceptable reading for a sensor on the vaccination cart.

9. The method of claim 6, further comprising receiving information regarding the number of remaining vaccination shots left in the refrigeration unit.

10. The method of claim 6, further comprising receiving information regarding any viability issues which may affect the integrity of the vaccination shots.

11. A server for optimizing, distributing and allocating vaccination shots comprising a processor operable to execute computer program instructions, a memory operable to store computer program instructions executable by the processor, and computer program instructions stored in the memory and executable to perform the steps of:
(a) receiving Provider information for a Provider;
(b) receiving a type of vaccine to be administered;
(c) reviewing a Provider profile or settings for the Provider;
(d) executing an algorithm to approximate the number of vaccination shots to deliver to the Provider based on the type of vaccine to be administered and the Provider profile or settings for the Provider; and
(e) creating a schedule to deliver the vaccination shots to the Provider;

12. The server of claim 11, further comprising computer program instructions stored in the memory and executable to perform the step of:
creating a Provider user account including the Provider information, wherein the Provider information includes a unique user id and password, a proper Provider level, and choosing profile settings.

13. The server of claim 12, further comprising computer program instructions stored in the memory and executable to perform the step of supplying the proper DEA identifiers.

14. The server of claim 11, wherein the step of creating a schedule further includes the step of:
identifying the number of Provider facilities to distribute vaccination shots to.

15. The server of claim 14, wherein the step of creating a schedule further includes the step of:
determining the schedules for each identified Provider facility.

16. The server of claim 15, wherein the step of creating a schedule further includes the step of:
specifying that a vaccination cart is to be provided along with the vaccination shots.

17. The server of claim 16, wherein the step of creating a schedule further includes the step of:

18. The server method of claim 16, further comprising computer program instructions stored in the memory and executable to perform the step of receiving notification that a Provider terminal has determined that there is an unacceptable reading for a sensor on the vaccination cart.

19. The server of claim 16, further comprising computer program instructions stored in the memory and executable to perform the step of receiving information regarding the number of remaining vaccination shots left in the refrigeration unit.

20. The server of claim 16, further comprising computer program instructions stored in the memory and executable to perform the step of receiving information regarding any viability issues which may affect the integrity of the vaccination shots.
